(19) United States
(12) Patent Application Publication
(10) Pub. No.: US 2026/0152515 A1
(43) Pub. Date: Jun. 4, 2026

(54) Tralopyril Derivative and Application Thereof

(71) Applicant: YANGTZE UNIVERSITY, Jingzhou (CN)

(72) Inventors: Zhihong XU, Jingzhou (CN); Wei ZHAO, Jingzhou (CN); Zhonglu ZHANG, Jingzhou (CN); Yue YAN, Jingzhou (CN)

(73) Assignee: YANGTZE UNIVERSITY, Jingzhou (CN)

(21) Appl. No.: 19/405,462

(22) Filed: Dec. 2, 2025

(30) Foreign Application Priority Data
Dec. 4, 2024 (CN) 202411767093.0
Jul. 22, 2025 (CN) 202511006533.5

(51) Int. Cl. C07F 9/572; A01N 43/36; A01N 57/34; A01P 5/00; C07D 207/34

(52) U.S. Cl. CPC C07F 9/572; A01N 43/36; A01N 57/34; A01P 5/00; C07D 207/34

(57) ABSTRACT

A Tralopyril derivative and use thereof are provided. The Tralopyril derivative may be used to control plant-parasitic nematodes, such as root-knot nematodes and cyst nematodes. The biological assay results show that, using the foliar spray method at a dosage only one twentieth of that used in the root-irrigation method, Tralopyril-β-aminobutyric acid exhibits higher potted-plant activity against root-knot nematodes in tomato, cowpea, and cucumber than cyclobutrifluram applied at the same concentration via the root-irrigation method. Tralopyril-β-aminobutyric acid also exhibits excellent control efficacy against tobacco root-knot nematodes when applied by both foliar spray and root-irrigation methods. Tralopyril-β-aminobutyric acid also exhibits certain control efficacy against cyst nematodes in soybean.

### CROSS REFERENCE TO THE RELATED APPLICATIONS

This application is based upon and claims priority to Chinese Patent Applications No. 202411767093.0, filed on Dec. 4, 2024, and No. 202511006533.5, filed on Jul. 22, 2025, the entire contents of which are incorporated herein by reference.

### TECHNICAL FIELD

The present invention relates to the technical field of pesticidal compounds, and in particular, to a Tralopyril derivative and use thereof.

### BACKGROUND

Plant-parasitic nematodes are one of the most important plant-infecting pathogens, and, due to characteristics of wide distribution, extremely small size, concealment underground, and severe damage to plant root systems, can cause yield reductions of major crops by about 12.3%, and losses can reach 60% under severe conditions. According to incomplete statistics, plant-parasitic nematodes cause agricultural losses of more than 150 billion USD annually worldwide. Root-knot nematodes (Meloidogyne spp.) are the nematodes with the largest number of species, the widest distribution, and the most severe damage. In addition to root-knot nematodes, the more severe plant-parasitic nematodes further include soybean cyst nematodes, sugar beet cyst nematodes, wheat seed-gall nematodes, sweet-potato stem nematodes, rice white-tip nematodes, millet nematodes, and pine-wood nematodes. Currently, chemical control is the primary approach for managing plant-parasitic nematodes, and traditional nematicides mainly include abamectin, emamectin benzoate, fosthiazate, oxamyl, and terbufos; however, resistance is severe and control efficacy is unsatisfactory. In recent years, the newly developed nematicides fluopyram and cyclobutrifluram have also rapidly developed resistance and are expensive.

Tralopyril is an arylpyrrole compound synthesized through structural modification of the natural antibiotic dihydropyrromycin and exhibits excellent insecticidal, acaricidal, fungicidal, and nematicidal activities; however, phytotoxicity to certain crops limits agricultural application of Tralopyril. At present, Tralopyril is mainly used as a pesticide intermediate for synthesizing the insecticide chlorfenapyr. Amino acids, as endogenous plant substances, are widely applied in the research and development of new pesticides due to the advantages of crop safety, environmental friendliness, and unique chemical structures. The most common amino acids in organisms are α-amino acids, and β-amino acids and γ-amino acids also exist. β-Aminobutyric acid (BABA) is a four-carbon non-protein amino acid present in plants, and is initially detected in root exudates of tomato subjected to excessive sunlight. As an inducer, BABA can induce systemic acquired resistance in plants against diseases caused by various pathogenic fungi, bacteria, viruses, and plant-parasitic nematodes. BABA is a broad-spectrum plant immunity inducer, which not only induces systemic resistance against pathogenic microorganisms in multiple plants, but also induces plant tolerance to abiotic stresses such as drought, high salinity, and extreme temperatures. Therefore, the introduction of β-aminobutyric acid into the Tralopyril structure to obtain a compound for controlling plant-parasitic nematode diseases while also possessing the ability to induce disease resistance has broad application prospects.

Triphenylphosphonium cations are the most common lipophilic cations and exhibit strong mitochondrial targeting ability. When coupled with pharmacophore groups, the triphenylphosphonium cations can penetrate cell membranes and drive accumulation of the pharmacophore groups in the negatively charged mitochondrial inner membrane. At present, the method of preparing targeted agents by coupling triphenylphosphonium cations with commercial drugs has been widely applied in medicine, but reports in agricultural applications are limited.

### SUMMARY

An objective of the present invention is to provide a Tralopyril derivative and use thereof, so as to solve the above technical problems.

To achieve the foregoing objective, the present invention provides the following technical solutions.

The present invention provides a Tralopyril derivative, which includes compound A and compound B;
the compound A includes compound 1 and compound 2:

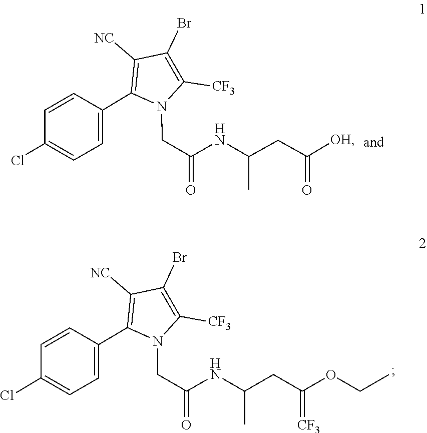

and
the compound B has the following structural formula:

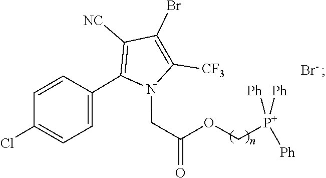

n=3-12.

Further, the compound B includes a compound having the following structural formula:

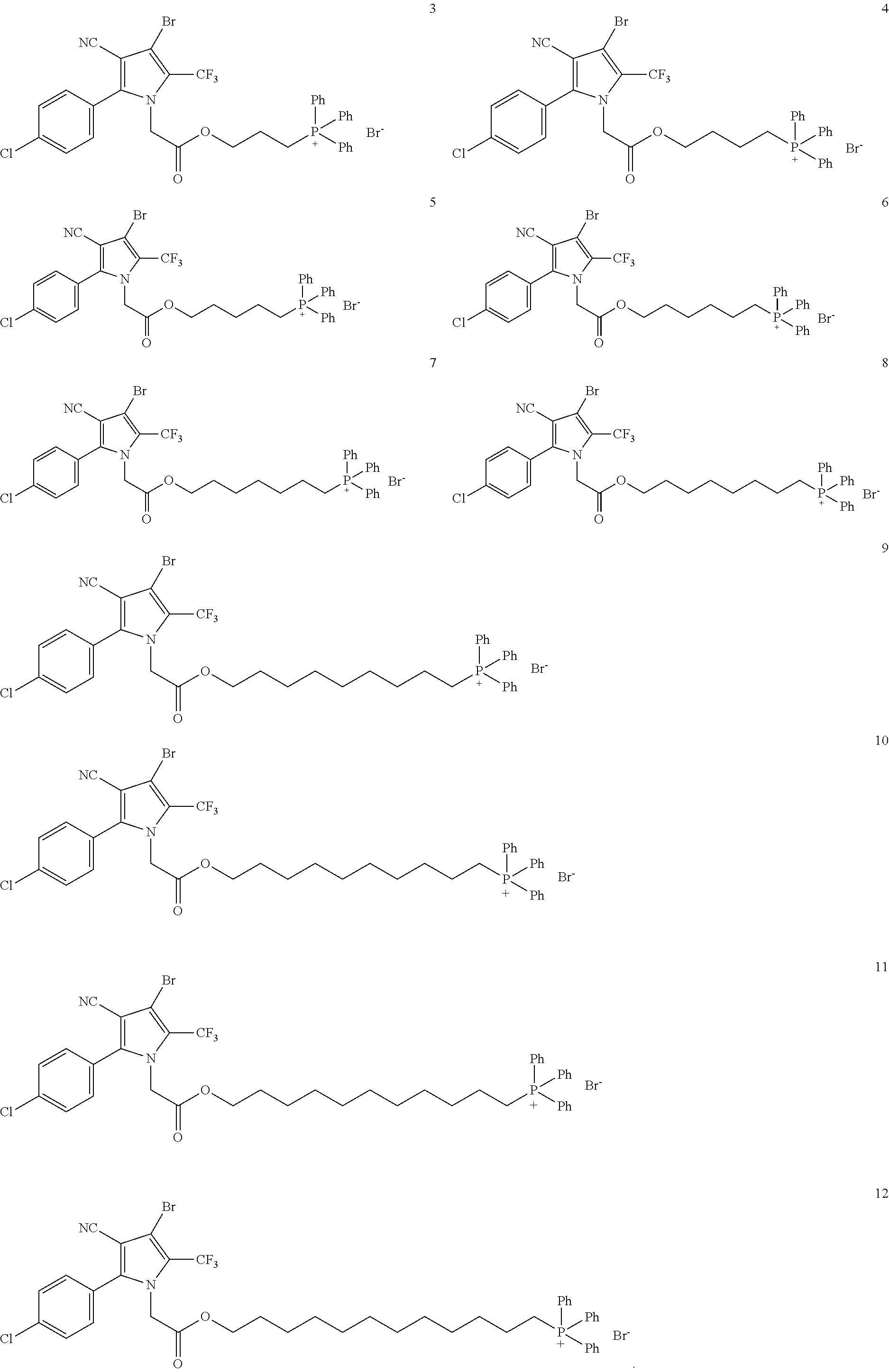

The present invention further provides use of the Tralopyril derivative in the control of nematode diseases, where the compound A is used for controlling root-knot nematodes and cyst nematodes in soybean, and the compound B is used for controlling root-knot nematodes.

Further, the root-knot nematode disease includes one or more of tomato root-knot nematode, cowpea root-knot nematode, cucumber root-knot nematode, and tobacco root-knot nematode.

The beneficial effects of the present invention are as follows.

The present invention uses Tralopyril as the parent structure and conjugates the Tralopyril with β-aminobutyric acid esters to obtain two new compounds with novel chemical structures, which can be used to control root-knot nematode diseases and soybean cyst nematode diseases. The biological assay results show that Tralopyril-β-aminobutyric acid applied by root-irrigation method (therapeutic) exhibits significantly superior control efficacy against tomato root-knot nematodes compared with fluopyram, and shows comparable efficacy to the novel nematicide cyclobutrifluram. When applied by foliar spray at a dosage only one twentieth of that used in the root-irrigation method, Tralopyril-β-aminobutyric acid exhibits higher potted-plant activity against root-knot nematodes of tomato, cowpea, and cucumber than the same concentration of the reference agent cyclobutrifluram (root-irrigation method). Tralopyril-β-aminobutyric acid also shows excellent control efficacy against tobacco root-knot nematodes by both foliar spray and root-irrigation methods. Tralopyril-β-aminobutyric acid also exhibits certain control efficacy against soybean cyst nematodes. The root-irrigation method requires a large application dosage, which contaminates soil and water, promotes soil compaction, poses high ecological risks, and is environmentally unfriendly. The foliar spray method is environmentally friendly and more ecologically safe, so that Tralopyril-β-aminobutyric acid has a high commercialization potential. Tralopyril-triphenylphosphonium derivatives also show good potted-plant activity against tomato root-knot nematodes when applied by root-irrigation method.

### BRIEF DESCRIPTION OF THE DRAWINGS

FIG. 1A to FIG. 1K show the control efficacy of compound 1 against tomato root-knot nematodes under different application methods;

FIG. 2A to FIG. 2K show the control efficacy of compound 1 against cowpea root-knot nematodes under different application methods;

FIG. 3A to FIG. 3K show the control efficacy of compound 1 against cucumber root-knot nematodes under different application methods; and

FIG. 4A to FIG. 4M show the control efficacy of compound 1 against tobacco root-knot nematodes under different application methods.

### DETAILED DESCRIPTION OF THE EMBODIMENTS

The present invention provides a Tralopyril derivative, which includes compound A and compound B;
the compound A includes compound 1 and compound 2:

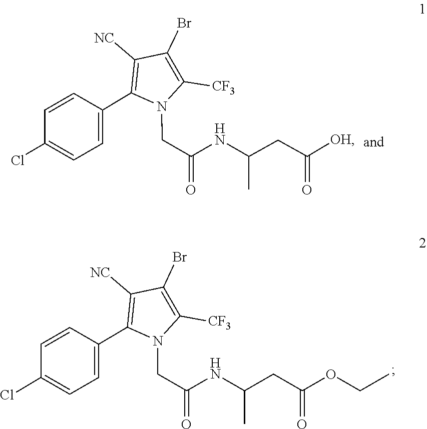

and
the compound B has the following structural formula:

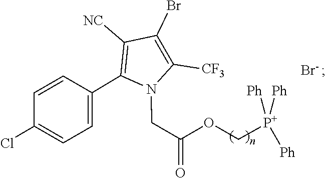

n=3-12.

In the present invention, the compound B includes a compound having the following structural formula:

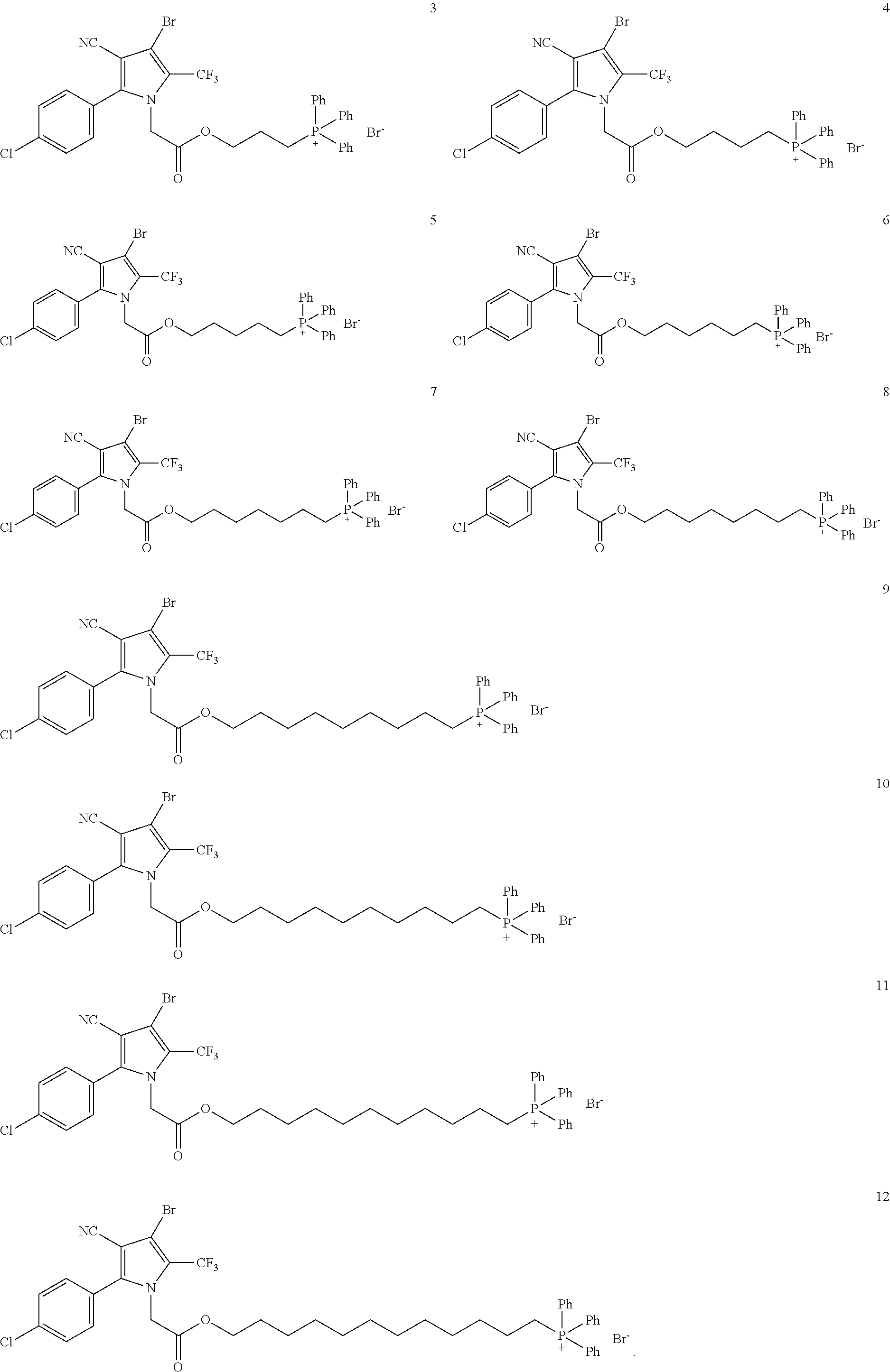

The present invention further provides use of the Tralopyril derivative in the control of nematode diseases, where the compound A is used for controlling root-knot nematodes and cyst nematodes in soybean, and the compound B is used for controlling root-knot nematodes.

In the present invention, the root-knot nematode disease includes one or more of tomato root-knot nematode, cowpea root-knot nematode, cucumber root-knot nematode, and tobacco root-knot nematode.

The technical solutions provided by the present invention will be described in detail below with reference to examples, which, however, should not be construed as limiting the scope of the present invention.

### Example 1

Provided is a Tralopyril derivative, where the molecular structures and physicochemical properties of compounds 1 to 12 are shown in Tables 1 and 2.

TABLE 1
Molecular structures and molecular formulas of compounds 1 to 12

| No. | Molecular structure | Molecular formula |
|---|---|---|
| 1 | 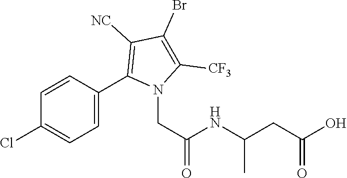 [SMILES: CC(CC(=O)O)NC(=O)Cn1c(-c2ccc(Cl)cc2)c(C#N)c(Br)c1C(F)(F)F] 1 | C₁₈H₁₄BrClF₃N₃O₃ |
| 2 | 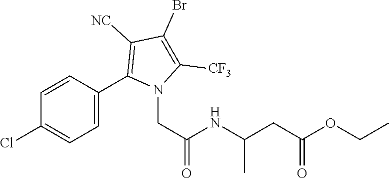 [SMILES: CCOC(=O)CC(C)NC(=O)Cn1c(-c2ccc(Cl)cc2)c(C#N)c(Br)c1C(F)(F)F] 2 | C₂₀H₁₈BrClF₃N₃O₃ |
| 3 | 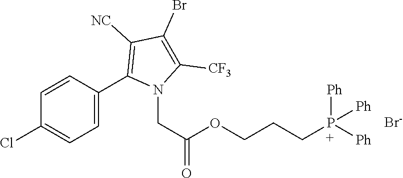 3 | C₃₅H₂₇Br₂ClF₃N₂O₂P |
| 4 | 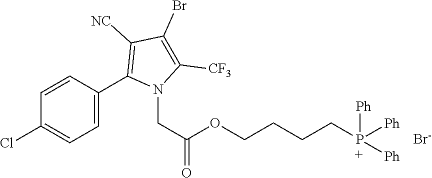 4 | C₃₆H₂₉Br₂ClF₃N₂O₂P |
| 5 | 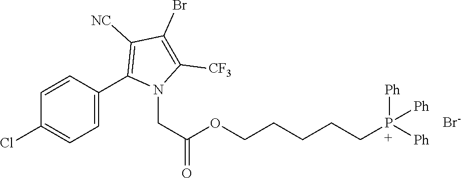 5 | C₃₇H₃₁Br₂ClF₃N₂O₂P |
| 6 | 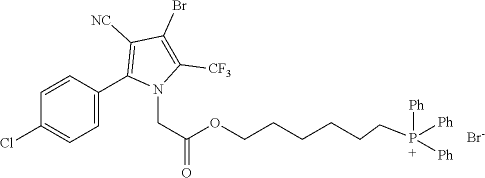 6 | C₃₈H₃₃Br₂ClF₃N₂O₂P |
| 7 | 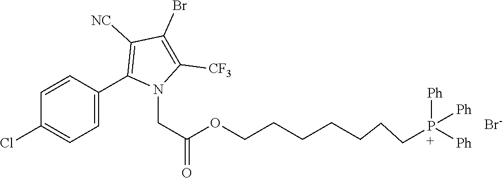 7 | C₃₉H₃₅Br₂ClF₃N₂O₂P |
| 8 | 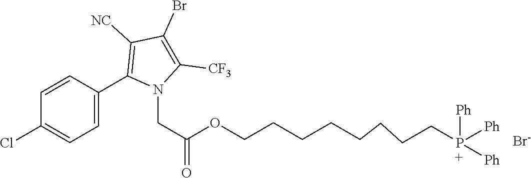 8 | C₄₀H₃₇Br₂ClF₃N₂O₂P |
| 9 | 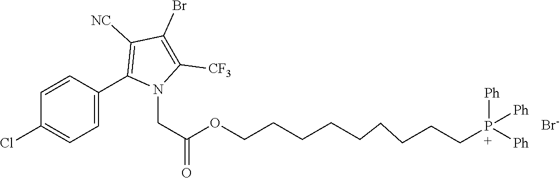 9 | C₄₁H₃₉B₁₂ClF₃N₂O₂P |
| 10 | 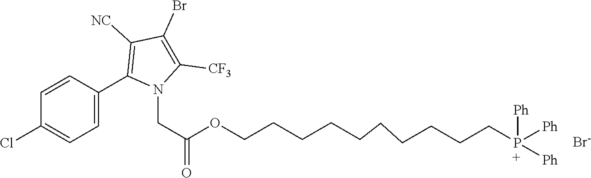 10 | C₄₂H₄₁Br₂ClF₃N₂O₂P |
| 11 | 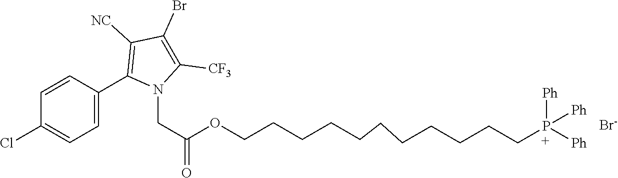 11 | C₄₃H₄₃Br₂ClF₃N₂O₂P |
| 12 | 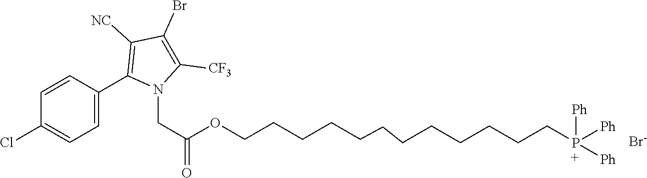 12 | C₄₄H₄₅B₁₂ClF₃N₂O₂P |

TABLE 2
¹H nuclear magnetic resonance (¹H NMR) data of compounds 1 to 12

| No. | ¹H NMR |
|---|---|
| 1 | ¹H NMR (400 MHz, DMSO) δ 12.24 (s, 1H), 8.27 (d, J = 8.0 Hz, 1H), |
| | 7.68 (d, J = 8.4 Hz, 2H), 7.55 (d, J = 8.4 Hz, 2H), 4.62-4.49 (m, 2H), 4.11-3.99 |
| | (m, 1H), 2.41-2.27 (m, 2H), 1.05 (d, J = 6.8 Hz, 3H). |
| 2 | ¹H NMR (400 MHz, CDCl₃) δ 7.49 (dd, J = 20.8, 8.6 Hz, 4H), 6.47 (d, J = 8.4 |
| | Hz, 1H), 4.54 (s, 2H), 4.39-4.27 (m, 1H), 4.17 (q, J = 7.2 Hz, 2H), 2.50 (t, J = |
| | 5.2 Hz, 2H), 1.29 (t, J = 7.2 Hz, 3H), 1.22 (d, J = 6.8 Hz, 3H). |
| 3 | ¹H NMR (400 MHz, CDCl₃) δ 7.93-7.77 (m, 9H), 7.71 (td, J = 7.7, 3.4 Hz, |
| | 6H), 7.52-7.46 (m, 2H), 7.40-7.34 (m, 2H), 4.82 (s, 2H), 4.57 (t, J = 6.1 Hz, |
| | 2H), 4.10-4.00 (m, 2H), 1.97 (s, 2H). |
| 4 | ¹H NMR (400 MHz, CDCl₃) δ 7.88-7.75 (m, 9H), 7.71 (dt, J = 7.8, 3.9 Hz, |
| | 6H), 7.49 (d, J = 8.2 Hz, 2H), 7.38 (d, J = 8.0 Hz, 2H), 4.84 (s, 2H), 4.52 (t, J = |
| | 6.1 Hz, 2H), 3.92 (qd, J = 8.4, 4.2 Hz, 2H), 2.04 (d, J = 6.1 Hz, 2H), 1.94 (t, J = |
| | 7.7 Hz, 2H) |
| 5 | ¹H NMR (400 MHz, CDCl₃) δ 7.81 (td, J = 13.5, 7.9 Hz, 9H), 7.68 (td, J = 7.7, |
| | 3.3 Hz, 6H), 7.48 (d, J = 8.1 Hz, 2H), 7.35 (d, J = 8.1 Hz, 2H), 4.68 (s, 2H), |
| | 4.11 (t, J = 5.9 Hz, 2H), 3.81 (td, J = 12.7, 6.3 Hz, 2H), 1.65 (ddt, J = 22.4, |
| | 14.9, 7.0 Hz, 6H). |
| 6 | ¹H NMR (400 MHz, CDCl₃) δ 7.79 (dt, J = 14.2, 6.6 Hz, 9H), 7.67 (tt, J = 6.5, |
| | 3.5 Hz, 6H), 7.50-7.43 (m, 2H), 7.33 (dd, J = 8.5, 2.3 Hz, 2H), 4.67 (s, 2H), |
| | 4.08 (d, J = 5.2 Hz, 2H), 3.71 (dd, J = 15.8, 9.1 Hz, 2H), 2.11 (s, 2H), 1.63 (dh, |
| | J = 14.2, 6.4 Hz, 6H) |
| 7 | ¹H NMR (400 MHz, CDCl₃) δ 7.90-7.74 (m, 9H), 7.71 (dt, J = 8.8, 4.4 Hz, |
| | 6H), 7.53-7.43 (m, 2H), 7.37 (dd, J = 8.7, 2.2 Hz, 2H), 4.70 (s, 2H), 4.13 (qd, |
| | J = 7.0, 3.4 Hz, 2H), 3.81 (td, J = 12.7, 6.8 Hz, 2H), 1.95 (s, 2H), 1.69 (q, J = |
| | 7.7 Hz, 2H), 1.60 (h, J = 7.0 Hz, 4H), 1.33 (t, J = 7.8 Hz, 2H) |
| 8 | ¹H NMR (400 MHz, CDCl₃) δ 7.78 (dt, J = 13.6, 6.7 Hz, 9H), 7.68 (td, J = 7.5, |
| | 3.3 Hz, 6H), 7.47 (d, J = 8.2 Hz, 2H), 7.35 (d, J = 8.5 Hz, 2H), 4.64 (s, 2H), |
| | 4.10 (t, J = 6.5 Hz, 2H), 3.67 (d, J = 13.4 Hz, 2H), 1.60 (s, 4H), 1.52 (d, J = 6.5 |
| | Hz, 2H), 1.23-1.17 (m, 6H) |
| 9 | ¹H NMR (400 MHz, CDCl₃) δ 7.83 (dddd, J = 11.7, 9.8, 7.2, 1.9 Hz, 9H), 7.71 |
| | (td, J = 7.8, 3.6 Hz, 6H), 7.51 (dq, J = 8.4, 2.2 Hz, 2H), 7.37 (dq, J = 8.6, 2.1 |
| | Hz, 2H), 4.66 (s, 2H), 4.18-4.11 (m, 2H), 3.75 (ddd, J = 12.8, 8.2, 4.3 Hz, |
| | 2H), 2.11 (d, J = 1.7 Hz, 2H), 1.69-1.51 (m, 6H), 1.23 (s, 6H) |
| 10 | ¹H NMR (400 MHz, CDCl₃) δ 7.89-7.77 (m, 9H), 7.71 (ddd, J = 8.6, 6.8, 3.4 |
| | Hz, 6H), 7.54-7.48 (m, 2H), 7.40-7.35 (m, 2H), 4.70-4.62 (m, 2H), 4.14 (t, |
| | J = 6.7 Hz, 2H), 3.77 (q, J = 9.0 Hz, 2H), 2.14-2.10 (m, 2H), 1.68-1.52 (m, |
| | 6H), 1.28 (s, 2H), 1.22 (d, J = 2.3 Hz, 6H) |
| 11 | ¹H NMR (400 MHz, CDCl₃) δ 7.83 (dddd, J = 11.7, 9.8, 7.2, 1.9 Hz, 9H), 7.71 |
| | (td, J = 7.8, 3.6 Hz, 6H), 7.51 (dq, J = 8.4, 2.2 Hz, 2H), 7.37 (dq, J = 8.6, 2.1 |
| | Hz, 2H), 4.66 (s, 2H), 4.18-4.11 (m, 2H), 3.75 (ddd, J = 12.8, 8.2, 4.3 Hz, |
| | 2H), 2.11 (d, J = 1.7 Hz, 2H), 1.69-1.51 (m, 6H), 1.23 (s, 6H) |
| 12 | ¹H NMR (400 MHz, CDCl₃) δ 7.81-7.73 (m, 9H), 7.67 (ddd, J = 8.5, 6.3, 3.4 |
| | Hz, 6H), 7.49-7.42 (m, 2H), 7.36-7.29 (m, 2H), 4.61 (s, 2H), 4.10-4.03 |
| | (m, 2H), 3.67-3.55 (m, 2H), 1.99 (s, 2H), 1.60-1.47 (m, 6H), 1.21-1.15 (m, |
| | 12H) |

### Experimental Example

The potted-plant assay for nematicidal activity was conducted using two test methods, namely root-irrigation and foliar spray:
For the root-irrigation method, the source of tomato root-knot nematodes was tomato plants affected by root-knot nematode disease in Honghe, Yunnan. Seven days after transplanting the test seedlings (tomato, cowpea, cucumber, and tobacco), tomato root pieces infested with root-knot nematodes were inoculated. The root-irrigation method was divided into protective and therapeutic root-irrigation: in the protective root-irrigation, the plants were treated by root-irrigation two days before inoculation; and in the therapeutic root-irrigation, the potted plants were treated by root-irrigation two days after inoculation. The application volume for root-irrigation was 200 mL per plant. After treatment with the formulations, the plants were cultivated at ambient temperature, and the severity of root-knot nematode disease in the untreated control group was evaluated.

The nematode source for the foliar spray method was the same as that used in the root-irrigation method. Seven days after transplanting the test seedlings (tomato, cowpea, cucumber, and tobacco), the potted plants were sprayed with the test formulations, with a spray volume of 10 mL per plant. Two days after spraying, tomato root pieces infested with root-knot nematodes were inoculated. The plants were then cultivated at ambient temperature, and the severity of root-knot nematode disease in the untreated control group was assessed.

Assessment method, timing, and frequency:
the severity of root-knot nematode infestation was evaluated.

Grading standard: Grade 0: Root system is healthy, with no root galls.

Grade 1: Root system has a small number of root galls, accounting for 1-25% of the entire root system.
Grade 3: Root system has a moderate number of root-knot nematodes, accounting for 26-50% of the entire root system.
Grade 5: Root system has a large number of root-knot nematodes, accounting for 51-75% of the entire root system.
Grade 7: Root system has an extremely large number of root-knot nematodes, accounting for 76-100% of the entire root system.

The control efficacy of the target compounds against tomato root-knot nematodes is shown in Table 3.

The inoculum of soybean cyst nematodes was obtained from the Henan Academy of Agricultural Sciences. The cyst nematodes were inoculated in soil. The assay method for soybean cyst nematodes was the same as that for root-knot nematodes.

TABLE 3
Control efficacy of compounds 1 and 2 against tomato root-knot
nematodes in potted plants (therapeutic root-irrigation method)

| | | Active ingredient | Disease | Control |
|---|---|---|---|---|
| | Formulation | dosage | index | efficacy |
| Treatment | name | (mg/plant) | (%) | (%) |
| 1 | 1 | 10 | 9.52 | 88.23 |
| 2 | 2 | 10 | 19.05 | 76.47 |
| 3 | Fluopyram | 15 | 57.14 | 29.41 |
| 4 | Abamectin | 3 | 7.14 | 91.18 |
| 5 | 450 g/L | 5.4 | 14.29 | 82.35 |
| | cyclobutrifluram | | | |
| | suspension | | | |
| | concentrate | | | |
| 6 | CK | — | 80.95 | — |

The bioassay results showed that compounds 1 and 2 at an active ingredient dose of 10 mg/plant exhibited control efficacy against tomato root-knot nematodes of 88.23% and 76.47%, respectively, which was significantly higher than that of the commercial reference agent fluopyram (active ingredient dose: 15 mg/plant, control efficacy: 29.41%); the control efficacy of compound 1 was slightly higher than that of the novel nematicide cyclobutrifluram (active ingredient dose: 5.4 mg/plant, control efficacy: 82.35%); and both compounds 1 and 2 exhibited lower control efficacy than abamectin (active ingredient dose: 3 mg/plant, control efficacy: 91.18%). At the same dose, compound 1 showed clearly higher control efficacy against tomato root-knot nematodes than compound 2.

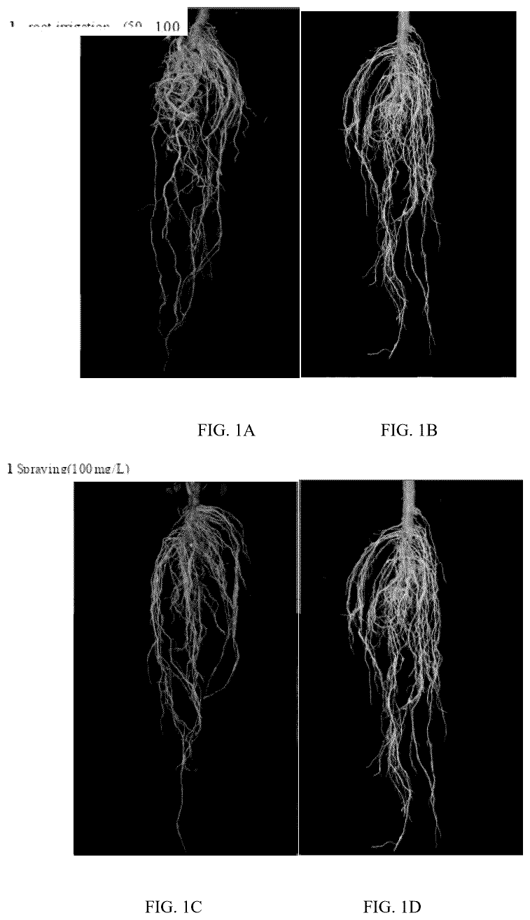

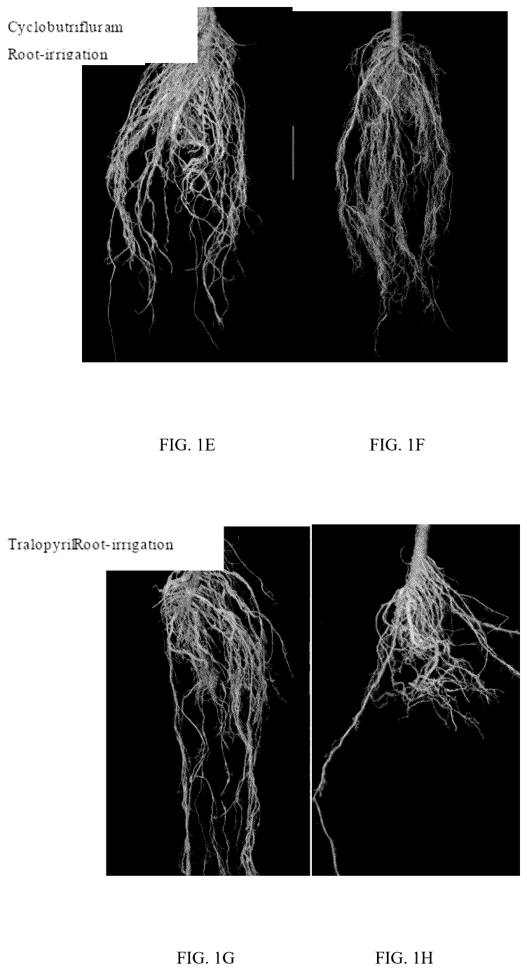

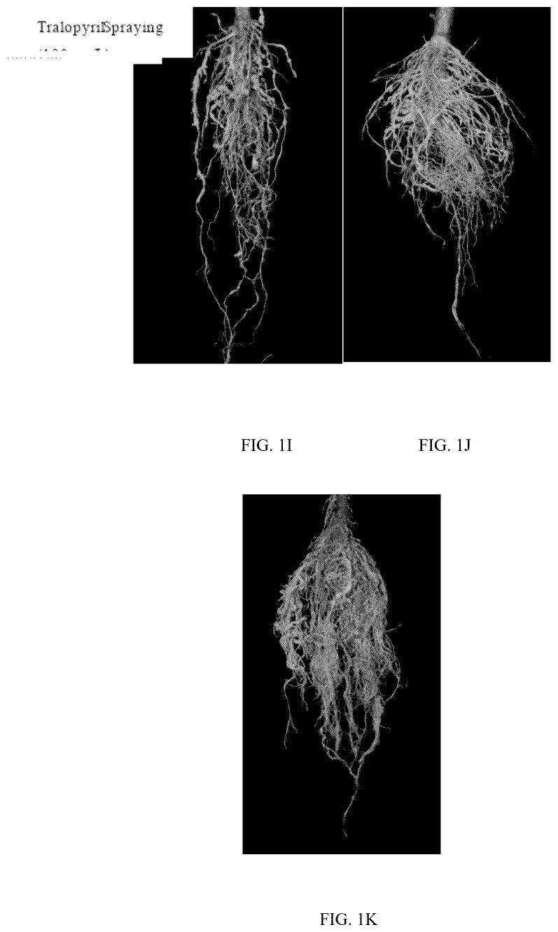

Further studies on the application method and control spectrum of compound 1 demonstrated that foliar spray also exhibited excellent control against tomato root-knot nematodes (control efficacy at 50 mg/L and 100 mg/L were 84.62% and 94.87%, respectively; detailed results are shown in Table 4 and FIGS. 1A-1K), which was significantly higher than that of cyclobutrifluram applied by therapeutic root-irrigation (control efficacy at 50 mg/L and 100 mg/L were 65.38% and 80.77%, respectively). At the same concentrations (50 and 100 mg/L), the actual application amount of compound 1 via foliar spray was only one-twentieth of that of cyclobutrifluram by therapeutic root-irrigation, while the control efficacy was higher. Moreover, compared with soil-applied therapeutic root-irrigation, foliar spray for the control efficacy against tomato root-knot nematodes is more environmentally friendly to soil, water, and the ecosystem.

TABLE 4
Control efficacy of compound 1 against tomato root-knot nematodes
in pot experiments using different application methods

| | | | | Control |
|---|---|---|---|---|
| | | Concentration | Disease | efficacy |
| Test agent | Application method | (mg/L) | index | (%) |
| Tralopyril | Therapeutic root-irrigation | 50 | 52.38 | 43.59 |
| Tralopyril | Therapeutic root-irrigation | 100 | 21.43 | 76.92 |
| Tralopyril | Spraying | 50 | 90.48 | 2.56 |
| Tralopyril | Spraying | 100 | 78.57 | 15.38 |
| 1 | Therapeutic root-irrigation | 50 | 9.52 | 89.74 |
| 1 | Therapeutic root-irrigation | 100 | 7.14 | 92.31 |
| 1 | Spraying | 50 | 14.29 | 84.62 |
| 1 | Spraying | 100 | 4.76 | 94.87 |
| Cyclobutrifluram | Therapeutic root-irrigation | 50 | 32.14 | 65.38 |
| Cyclobutrifluram | Therapeutic root-irrigation | 100 | 17.86 | 80.77 |
| CK | Root-irrigation | — | 92.86 | — |

It was found in further studies that compound 1 not only exhibited excellent control against tomato root-knot nematodes, but also demonstrated superior control against cucumber root-knot nematodes and cowpea root-knot nematodes.

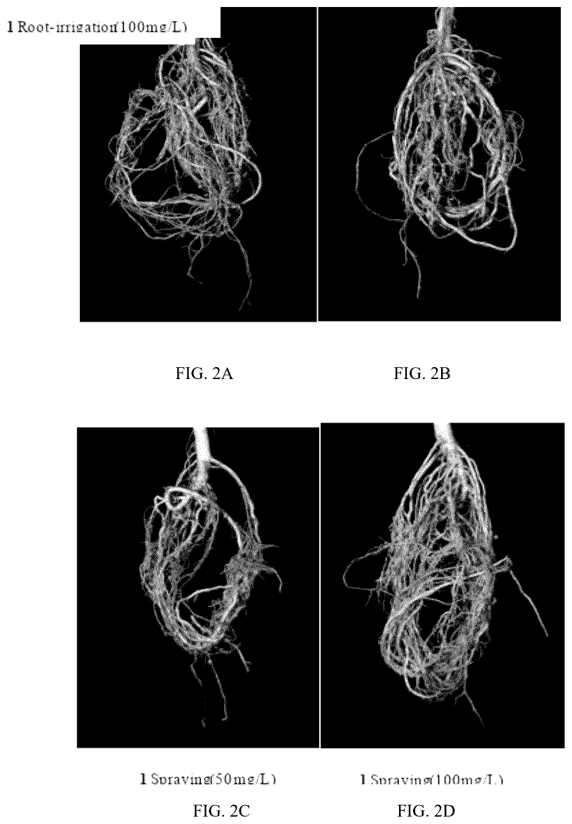

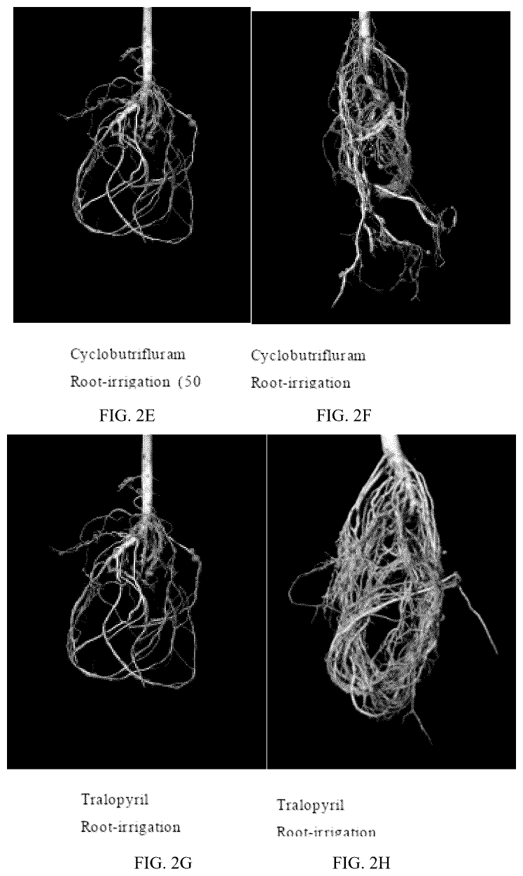

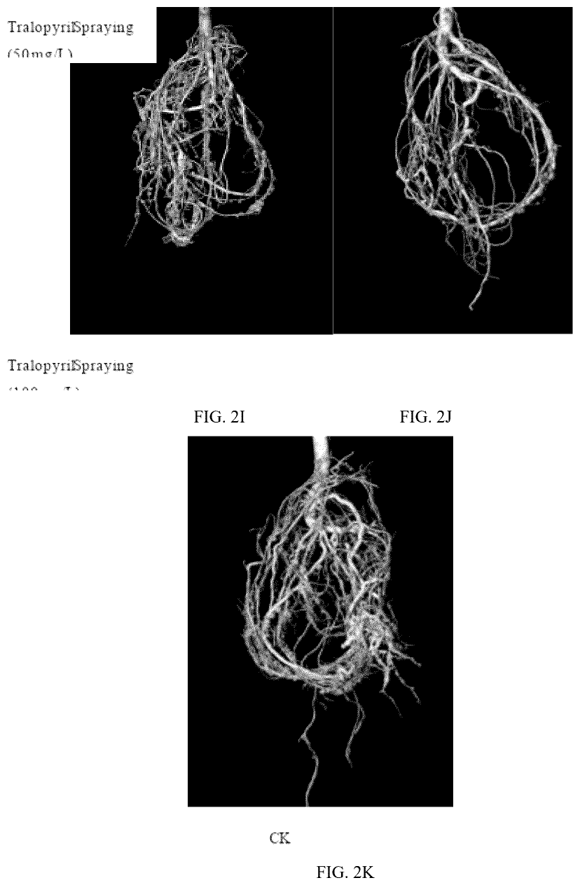

Compound 1 applied by foliar spray also exhibited excellent efficacy against cowpea root-knot nematodes (control efficacies at 50 mg/L and 100 mg/L were 82.14% and 96.43%, respectively; see detailed results in Table 5 and FIGS. 2A-2K), significantly higher than the control efficacy of cyclobutrifluram applied by therapeutic root-irrigation (control efficacies at 50 mg/L and 100 mg/L were 40.48% and 64.29%, respectively). At the same concentrations (50 and 100 mg/L), because foliar spray was used for compound 1 to control cowpea root-knot nematodes, the actual applied amount was only one-twentieth of cyclobutrifluram (therapeutic root-irrigation), yet the control efficacy was higher than that of cyclobutrifluram.

TABLE 5
Control efficacy of compound 1 against cowpea root-knot nematodes
in pot experiments using different application methods

| | | | | Control |
|---|---|---|---|---|
| | | Concentration | Disease | efficacy |
| Test agent | Application method | (mg/L) | index | (%) |
| Tralopyril | Therapeutic root-irrigation | 50 | 54.76 | 45.24 |
| Tralopyril | Therapeutic root-irrigation | 100 | 30.95 | 69.05 |
| Tralopyril | Spraying | 50 | 95.24 | 4.76 |
| Tralopyril | Spraying | 100 | 90.48 | 9.52 |
| 1 | Therapeutic root-irrigation | 50 | 10.71 | 89.29 |
| 1 | Therapeutic root-irrigation | 100 | 7.14 | 92.86 |
| 1 | Spraying | 50 | 17.86 | 82.14 |
| 1 | Spraying | 100 | 3.57 | 96.43 |
| Cyclobutrifluram | Therapeutic root-irrigation | 50 | 59.52 | 40.48 |
| Cyclobutrifluram | Therapeutic root-irrigation | 100 | 35.71 | 64.29 |
| CK | Root-irrigation | — | 100 | — |

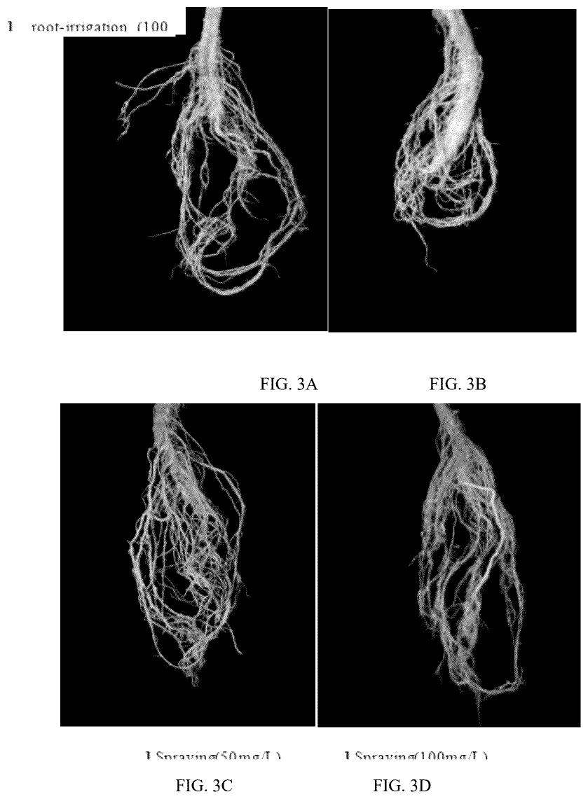

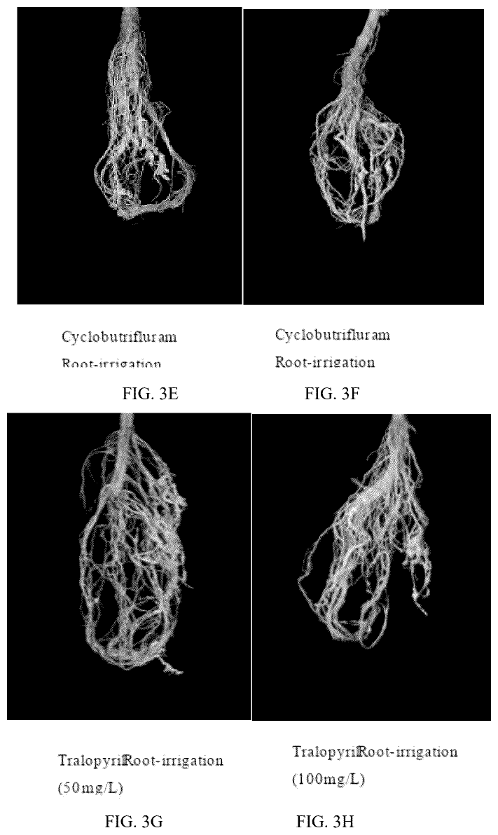

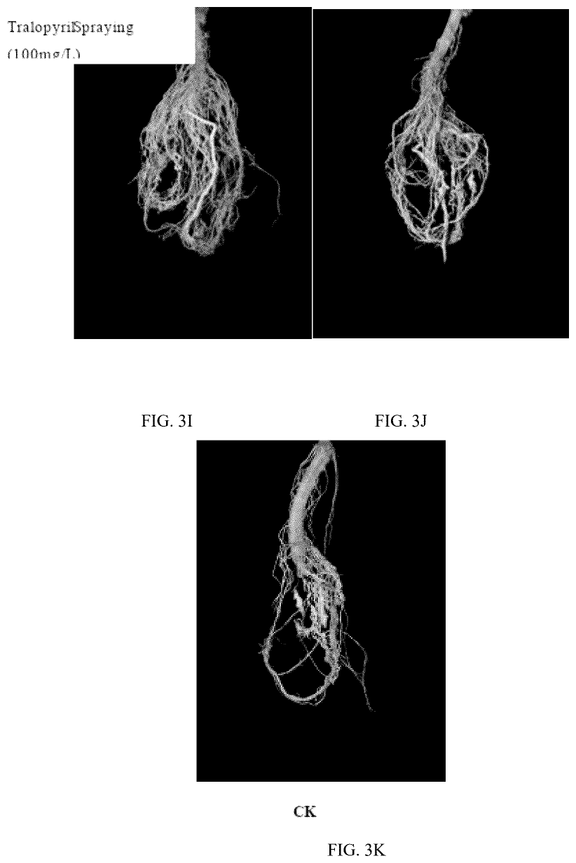

Compound 1 applied by foliar spray also exhibited excellent control against cucumber root-knot nematodes (control efficacies at 50 mg/L and 100 mg/L were 86.84% and 94.74%, respectively), significantly higher than the control efficacies of cyclobutrifluram applied by therapeutic root-irrigation (control efficacies at 50 mg/L and 100 mg/L were 23.81% and 61.90%, respectively; see Table 6 and FIGS. 3A-3K). At the same concentrations (50 and 100 mg/L), because foliar spray was used for compound 1 to control cucumber root-knot nematodes, the actual applied amount was only one-twentieth of cyclobutrifluram (therapeutic root-irrigation), yet the control efficacy was higher than that of cyclobutrifluram.

TABLE 6
Control efficacy of compound 1 against cucumber root-knot nematodes
in pot experiments using different application methods

| | | | | Control |
|---|---|---|---|---|
| | | Concentration | Disease | efficacy |
| Test agent | Application method | (mg/L) | index | (%) |
| Tralopyril | Therapeutic root-irrigation | 50 | 57.14 | 42.86 |
| Tralopyril | Therapeutic root-irrigation | 100 | 28.57 | 71.43 |
| Tralopyril | Spraying | 50 | 80.95 | 19.05 |
| Tralopyril | Spraying | 100 | 76.19 | 23.81 |
| 1 | Therapeutic root-irrigation | 50 | 9.52 | 89.47 |
| 1 | Therapeutic root-irrigation | 100 | 7.14 | 92.11 |
| 1 | Spraying | 50 | 11.90 | 86.84 |
| 1 | Spraying | 100 | 4.76 | 94.74 |
| Cyclobutrifluram | Therapeutic root-irrigation | 50 | 76.19 | 23.81 |
| Cyclobutrifluram | Therapeutic root-irrigation | 100 | 38.10 | 61.90 |
| CK | Root-irrigation | — | 100 | — |

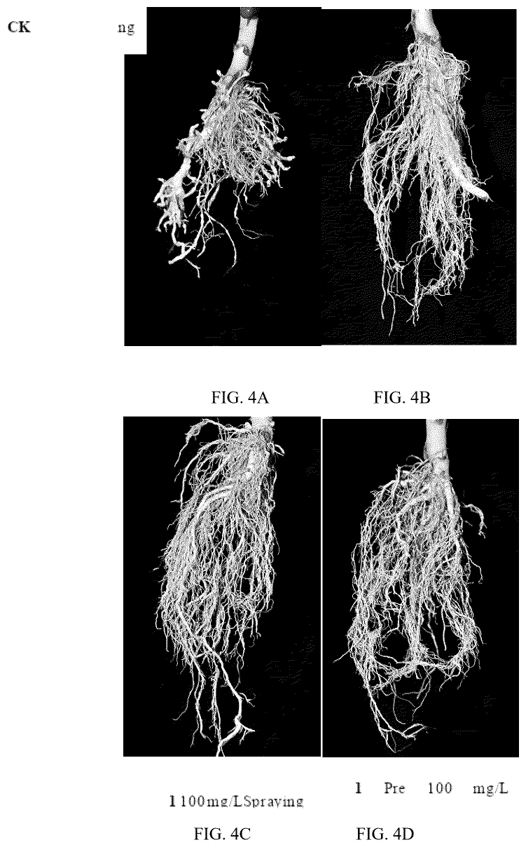

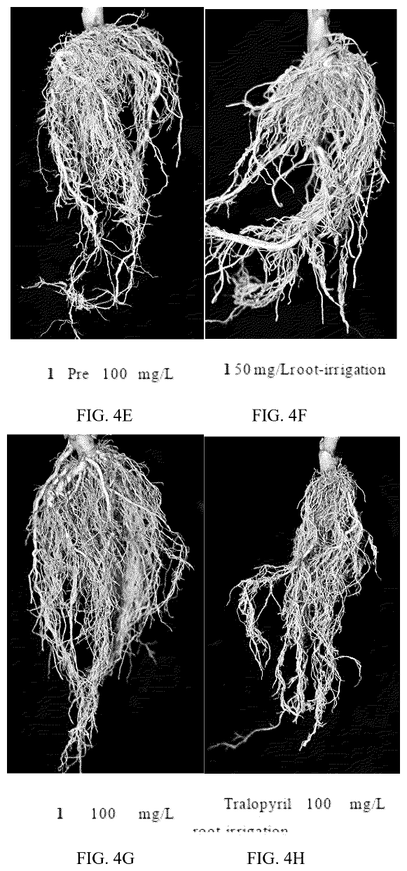

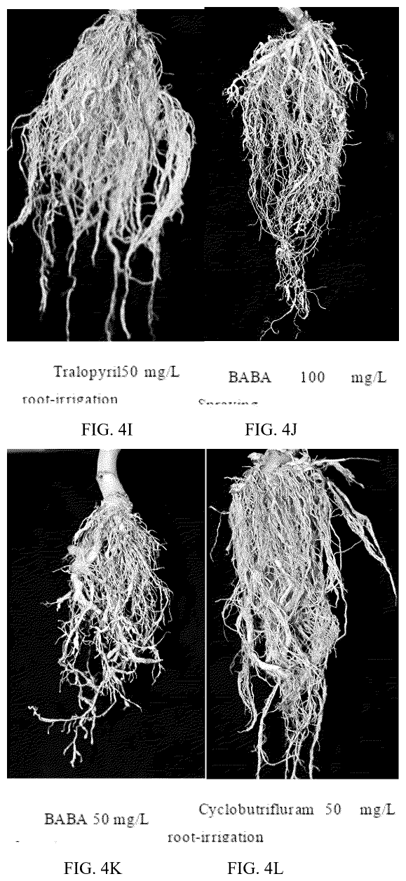

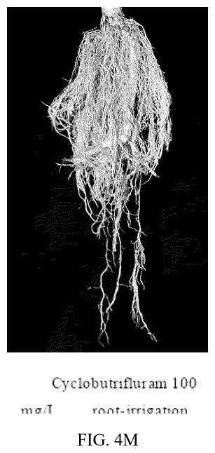

Compound 1 applied by foliar spray also exhibited excellent control against tobacco root-knot nematodes (40 days after treatment, control efficacies at 50 mg/L and 100 mg/L were 85.71% and 89.29%, respectively), slightly lower than those of cyclobutrifluram applied by therapeutic root-irrigation (control efficacies at 50 mg/L and 100 mg/L were 92.86% and 96.43%, respectively; see Table 7 and FIGS. 4A-4M).

TABLE 7
Control efficacy of compound 1 against tobacco root-knot nematodes
in pot experiments using different application methods

| | | | | | Control |
|---|---|---|---|---|---|
| | Concentration | | | Disease | efficacy |
| Treatment No. | (mg/L) | | Application method | index | % |
| 1 | 50 | P | Therapeutic root-irrigation | 35.71 | 64.29 |
| 1 | 100 | P | Therapeutic root-irrigation | 28.57 | 71.43 |
| 1 | 50 | P | Protective root-irrigation | 10.71 | 89.29 |
| 1 | 100 | P | Protective root-irrigation | 7.14 | 92.86 |
| 1 | 50 | P | Spraying | 14.29 | 85.71 |
| 1 | 100 | P | Spraying | 10.71 | 89.29 |
| Tralopyril | 50 | P | Therapeutic root-irrigation | 28.57 | 71.43 |
| Tralopyril | 100 | P | Therapeutic root-irrigation | 14.29 | 85.71 |
| Cyclobutrifluram | 50 | P | Therapeutic root-irrigation | 7.14 | 92.86 |
| Cyclobutrifluram | 100 | P | Therapeutic root-irrigation | 3.57 | 96.43 |
| BABA | 50 | P | Spraying | 100.00 | 0.00 |
| BABA | 100 | P | Spraying | 100.00 | 0.00 |

| | | | | |
|---|---|---|---|---|
| CK | — | — | 100.00 | 0.00 |

Compound 1 applied by foliar spray also exhibited certain control efficacy against soybean cyst nematodes (at 42 days after treatment, the control efficacies at 50 mg/L and 100 mg/L were 64.29% and 71.43%, respectively). The control efficacy of compound 1 applied by root-irrigation (at 50 mg/L and 100 mg/L, 78.57% and 85.71%, respectively) was higher than that of foliar spray, but the control efficacies of compound 1 applied by foliar spray and via root-irrigation were lower than that of the commercial reference agent cyclobutrifluram applied by root-irrigation (at 50 mg/L and 100 mg/L, 92.86% and 96.43%, respectively). The results are shown in Table 8.

TABLE 8
Control efficacy of compound 1 against soybean cyst nematodes
in pot experiments using different application methods

| | | | | | Control |
|---|---|---|---|---|---|
| | Concentration | | | Application | efficacy |
| Treatment No. | (mg/L) | | Application method | time | % |
| 1 | 50 | P | Protective root-irrigation | 2 days before | 78.57 |
| | | | | inoculation | |
| 1 | 100 | P | Protective root-irrigation | 2 days before | 85.71 |
| | | | | inoculation | |
| 1 | 50 | P | Spraying | 2 days before | 64.29 |
| | | | | inoculation | |
| 1 | 100 | P | Spraying | 2 days before | 71.43 |
| | | | | inoculation | |
| Tralopyril | 50 | P | Therapeutic root-irrigation | 2 days after | 42.86 |
| | | | | inoculation | |
| Tralopyril | 100 | P | Therapeutic root-irrigation | 2 days after | 78.57 |
| | | | | inoculation | |
| Cyclobutrifluram | 50 | P | Therapeutic root-irrigation | 2 days after | 92.86 |
| | | | | inoculation | |
| Cyclobutrifluram | 100 | P | Therapeutic root-irrigation | 2 days after | 96.43 |
| | | | | inoculation | |
| BABA | 50 | P | Spraying | 2 days before | 0.00 |
| | | | | inoculation | |
| BABA | 100 | P | Spraying | 2 days before | 0.00 |
| | | | | inoculation | |

| | | | |
|---|---|---|---|
| CK | — | — | 0.00 |

Compounds 3-12 applied by root-irrigation also exhibited certain control efficacy against tomato root-knot nematodes (for compound 8, the control efficacies at 50 mg/L and 100 mg/L were 63.33% and 76.67%, respectively), slightly lower than the control efficacy of the commercial reference agent cyclobutrifluram applied by root-irrigation (61.1186% and 72.22% at 50 mg/L and 100 mg/L, respectively). The results are shown in Table 9.

TABLE 9
Control efficacy of Tralopyril-triphenylphosphonium derivatives against
tomato root-knot nematodes in pot experiments (root-irrigation)

| | | | Control |
|---|---|---|---|
| | | Disease | efficacy |
| Compound | Concentration | index | % |

| | | | | |
|---|---|---|---|---|
| 3 | 50 | mg/L | 71.43 | 16.67 |
| | 100 | mg/L | 61.90 | 27.78 |
| 4 | 50 | mg/L | 57.14 | 33.33 |
| | 100 | mg/L | 38.78 | 54.76 |
| 5 | 50 | mg/L | 77.14 | 10.00 |
| | 100 | mg/L | 54.29 | 36.67 |
| 6 | 50 | mg/L | 77.14 | 10.00 |
| | 100 | mg/L | 65.71 | 23.33 |
| 7 | 50 | mg/L | 66.67 | 22.22 |
| | 100 | mg/L | 57.14 | 33.33 |
| 8 | 50 | mg/L | 31.43 | 63.33 |
| | 100 | mg/L | 20.00 | 76.67 |
| 9 | 50 | mg/L | 48.57 | 43.33 |
| | 100 | mg/L | 42.86 | 50.00 |
| 10 | 50 | mg/L | 60.00 | 30.00 |
| | 100 | mg/L | 42.86 | 50.00 |
| 11 | 50 | mg/L | 64.29 | 25.00 |
| | 100 | mg/L | 50.00 | 41.67 |
| 12 | 50 | mg/L | 66.67 | 22.22 |
| | 100 | mg/L | 54.29 | 36.67 |
| Tralopyril | 50 | mg/L | 31.43 | 63.33 |
| | 100 | mg/L | 25.71 | 70 |
| Cyclobutrifluram | 50 | mg/L | 35.71 | 61.11 |
| | 100 | mg/L | 23.81 | 72.22 |

| | | | |
|---|---|---|---|
| CK | — | 85.71 | — |

According to the above examples, the present invention provides a Tralopyril derivative and use thereof. The present invention provides a series of novel compounds by using Tralopyril as a parent structure and splicing active substructures with various substituted β-aminobutyric acid derivatives. The resulting compounds exhibit novel chemical structures and can be used to control plant-parasitic nematodes, including root-knot nematodes (tomato root-knot nematode, cowpea root-knot nematode, and cucumber root-knot nematode) and soybean cyst nematode. The biological assay results show that, when applied by root-irrigation, Tralopyril-β-aminobutyric acid exhibit the control efficacy against tomato root-knot nematodes, which is significantly higher than that of the commercial reference agent fluopyram and comparable to that of the novel cyclobutrifluram. Moreover, when applied by foliar spray, Tralopyril-β-aminobutyric acid exhibits superior control efficacy against tomato root-knot nematode, cowpea root-knot nematode, and cucumber root-knot nematode compared with the same concentration of the reference agent cyclobutrifluram applied by root-irrigation. The actual application amount of Tralopyril-β-aminobutyric acid by foliar spray is only one-twentieth of that used in root-irrigation, yet the Tralopyril-β-aminobutyric acid still exhibits higher nematicidal activity than the reference agent. Furthermore, the root-irrigation method requires a large application dosage, which contaminates soil and water, promotes soil compaction, poses high ecological risks, and is environmentally unfriendly. The foliar spray method is environmentally friendly and more ecologically safe.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A Tralopyril derivative, comprising compound A and compound B; wherein the compound A comprises compound 1 and compound 2: 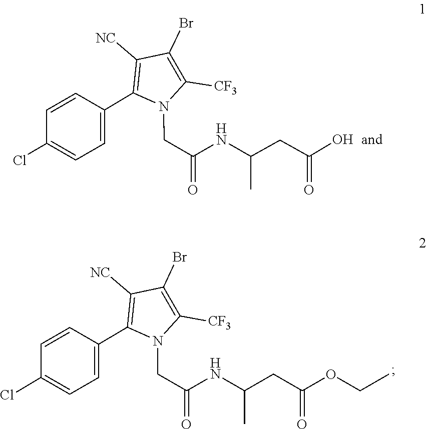
and
the compound B has the following structural formula: 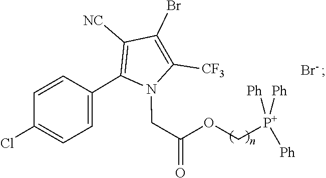
n=3-12.

2. The Tralopyril derivative according to claim 1, wherein the compound B comprises a compound having the following structural formula:

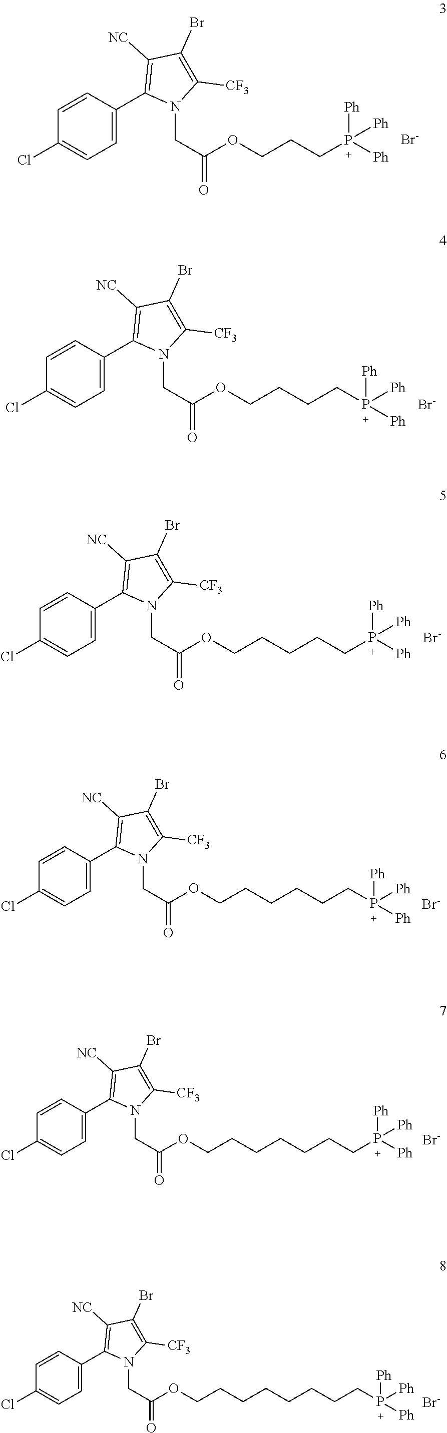

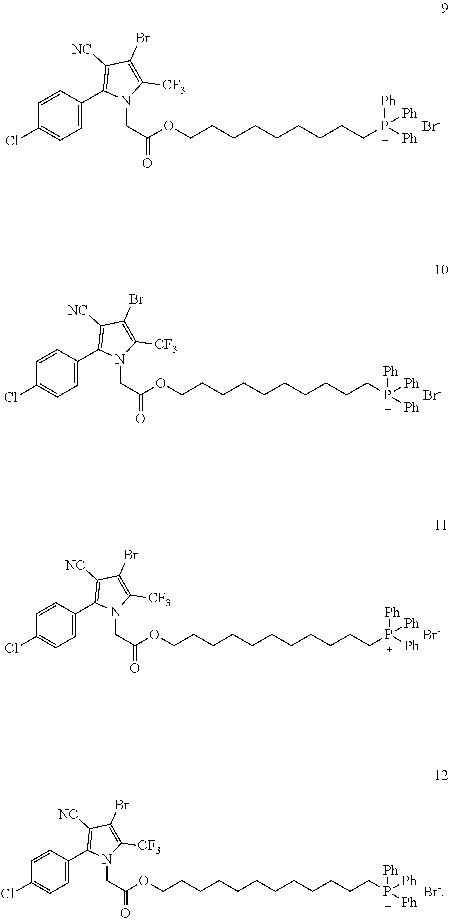

3. A method for controlling nematode diseases, comprising using the Tralopyril derivative according to claim 1, wherein the compound A is used for controlling root-knot nematodes and cyst nematodes in soybean, and the compound B is used for controlling the root-knot nematodes.

4. The method according to claim 3, wherein a root-knot nematode disease comprises one or more of tomato root-knot nematode, cowpea root-knot nematode, cucumber root-knot nematode, and tobacco root-knot nematode.

5. The method according to claim 3, wherein the compound B comprises a compound having the following structural formula:

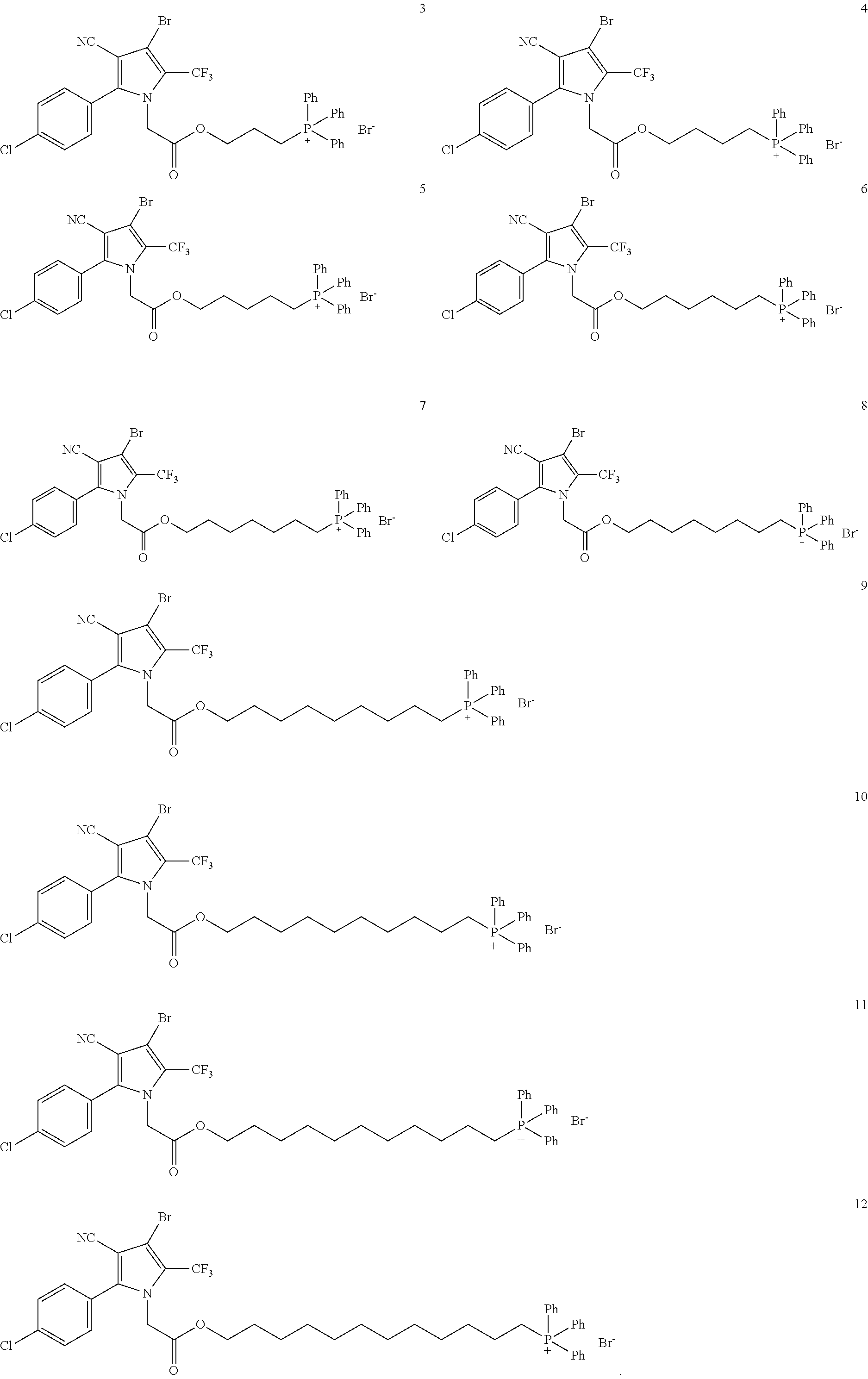

6. The method according to claim 5, wherein a root-knot nematode disease comprises one or more of tomato root-knot nematode, cowpea root-knot nematode, cucumber root-knot nematode, and tobacco root-knot nematode.
